(19) United States
(12) Patent Application Publication
(10) Pub. No.: US 2026/0354784 A1
(43) Pub. Date: Nov. 12, 2026

(54) X-RAY DOSE DETERMINATION APPARATUS

(71) Applicant: KONINKLIJKE PHILIPS N.V., EINDHOVEN (NL)

(72) Inventors: JOËL VALENTIN STADELMANN, MOSCOW (RU); ALEXANDER SELIVANOV, UNTERSCHLEIßHEIM (DE); HEINRICH SCHULZ, HAMBURG (DE)

(21) Appl. No.: 19/134,891

(22) PCT Filed: Dec. 1, 2023
(86) PCT No.: PCT/EP2023/083907

§ 371 Date: Jun. 2, 2025

(30) Foreign Application Priority Data
Dec. 9, 2022 (RU) 2022132294

(51) Int. Cl. A61B 6/00

(52) U.S. Cl. CPC A61B 6/544; A61B 6/488

(57) ABSTRACT

The present invention relates to an X-ray dose determination apparatus (10), comprising:—an input unit (20);—a processing unit (30); and—an output unit (40); wherein the input unit is configured to provide a visible or infrared image of a patient to the processing unit; wherein the processing unit is configured to determine a silhouette of the patient in the visible or infrared image of the patient; wherein the processing unit is configured to determine a thickness of the patient comprising utilization of the silhouette of the patient in the visible or infrared image of the patient; wherein the processing unit is configured to determine an X-ray dose for an X-ray examination of the patient comprising utilization of the thickness of the patient; and/or the processing unit is configured to determine that an atypical X-ray dose for the X-ray examination of the patient is required comprising utilization of the thickness of the patient; and wherein the output unit is configured to output an indication of the X-ray dose for the X-ray examination of the patient and/or the output unit is configured to output an indication that an atypical X-ray dose for the X-ray examination of the patient is required.

### FIELD OF THE INVENTION

The present invention relates to an X-ray dose determination apparatus, an X-ray dose determination system, an X-ray system, an X-ray dose determination method, a computer program element, and a computer readable medium.

### BACKGROUND OF THE INVENTION

The X-ray imaging of patients poses problem, where the imaging of overweight patient is consistently more complicated, than that of fitter patients. This is in part caused by difficulties in positioning the detector or achieving a correct field of view. However, a recurrent problem is that of the exposure. The radiation dose, that is emitted in the taking of an X-Ray (CXR) must be carefully calibrated in order to achieve a compromise between image quality and patient radiation exposure.

In the case of under-exposure, even though the image can be amplified and rescaled to present a good grayscale display, the noise in the image is likewise amplified, resulting in a noisy and grainy image in many cases. When such a non-optimal radiation dose applied the poor quality of the image often necessitates a retake—see for example: W. Huda, J. A. Seibert, K. Ogden, E. Gingold, R. Schaetzing, Physics Teaching File for Radiology Residents, Upstate Medical University, https://www.upstate.edu/radiology/education/rsna/radiography/issues.php.

Except for extreme cases, over-exposed images are usually of excellent radiographic quality with high contrast, and low noise. However, the patient in this situation unfortunately has received needless radiation exposure. In some cases, a three to five times overexposure or more can happen—see for example: W. Huda, J. A. Seibert, K. Ogden, E. Gingold, R. Schaetzing, Physics Teaching File for Radiology Residents, Upstate Medical University, https://www.upstate.edu/radiology/education/rsna/radiography/issues.php, and S. B. Gay, J. Olazagasti, J. W. Higginbotham, A. Gupta, A. Wurm, J. Nguyen, Introduction to Chest Radiology, University of Virginia Health Sciences Center, Department of Radiology, available online: https://introductiontoradiology.net/courses/rad/cxr/index.html#.

The radiation exposure is mostly a function of two factors, the X-ray tube's acceleration voltage, expressed in kV (kilo-Volts), and the exposure time product (radiation intensity), expressed in mAs. The kV controls the wavelength of the photons emitted by the tube and thus how they traverse the different tissues. Therefore, the kV controls the contrast of the images. Since contrast can be digitally enhanced, the kV parameter is less important in modern digital imaging systems—see for example L-C. Chen, G. Papandreou, F. Schroff, H. Adam, Rethinking Atrous Convolution for Semantic Image Segmentation, arXiv cs.CV, available online: https://arxiv.org/abs/1706.05587v3.

The mAs is a function of time, that controls the number of photons hitting the detector. Therefore, high mAs produce over-exposed images, while low mAs produces images, that are highly corrupted by noise—see for example L-C. Chen, G. Papandreou, F. Schroff, H. Adam, Rethinking Atrous Convolution for Semantic Image Segmentation, arXiv cs.CV, available online: https://arxiv.org/abs/1706.05587v3.

Other parameters such as collimation, and distance to patient can also influence the quality of the produced X-ray, mostly on the Field of View, and in the case of collimation on tissue contrast as well.

There is a need to address these problems.

### SUMMARY OF THE INVENTION

It would be advantageous to have an improved technique to help determine the X-ray dose for an X-ray image acquisition. The object of the present invention is solved with the subject matter of the independent claims, wherein further embodiments are incorporated in the dependent claims.

In a first aspect, there is provided an X-ray dose determination apparatus, comprising:
an input unit;
a processing unit; and
an output unit.

The input unit is configured to provide a visible or infrared image of a patient to the processing unit. The processing unit is configured to determine a silhouette of the patient in the visible or infrared image of the patient. The processing unit is configured to determine a thickness of the patient comprising utilization of the silhouette of the patient in the visible or infrared image of the patient. The processing unit is configured to determine an X-ray dose for an X-ray examination of the patient comprising utilization of the thickness of the patient; and/or the processing unit is configured to determine that an atypical X-ray dose for the X-ray examination of the patient is required comprising utilization of the thickness of the patient. The output unit is configured to output an indication of the X-ray dose for the X-ray examination of the patient and/or the output unit is configured to output an indication that an atypical X-ray dose for the X-ray examination of the patient is required.

In this manner a simple 2D image of a person acquired by a camera can be used to determine a thickness of a patient from which a correct X-ray dose can be determined and/or an indication given to an operator that an atypical or non-typical X-ray dose will be required. In this manner, for patients who are larger than normal and/or have more body fat can be accommodated where the correct X-ray dose can be given to enable a resultant X-ray with the required contrast to be acquired and mitigate overexposure or underexposure of the patient.

In other words, a 2D image of a patient is used to determine the patient's morphology from which an X-ray dose for an X-ray examination can be determined.

To put this another way a simple camera, such as an RGB camera, can be used to acquire an image and the patient's silhouette is evaluated from this image and this used to determine a thickness of the patient from which a correct X-ray dose can be determined.

In an example, the determination of the silhouette of the patient in the visible or infrared image of the patient comprises implementation by the processing unit of a segmentation machine learning algorithm to analyse the visible or infrared image of the patient to determine a segmentation mask representative of the silhouette of the patient in the visible or infrared image of the patient.

In an example, the segmentation machine learning algorithm was trained on a plurality of visible or infrared images of one or more persons and an associated plurality of segmentation masks obtained from annotation of an outline of the one or more persons, each visible or infrared image has an image of one person.

In an example, the processing unit is configured to determine a contour of the silhouette of the patient in the visible or infrared image of the patient comprising utilization of the silhouette of the patient in the visible or infrared image of the patient. The determination of the thickness of the patient can then comprise utilization of the contour of the silhouette of the patient in the visible or infrared image of the patient.

In an example, the processing unit is configured to determine a plurality of feature points of the silhouette of the patient in the visible or infrared image of the patient that represent the contour of the silhouette of the patient in the visible or infrared image of the patient. The determination of the thickness of the patient can then comprise utilization of the plurality of feature points of the silhouette of the patient in the visible or infrared image of the patient.

In an example, the determination of the plurality of feature points comprises a determination of a plurality of turning points at one or more boundaries of the silhouette of the patient in the visible or infrared image of the patient, and each turning point defines a feature point.

In an example, the determination of a turning point comprises a determination of a first direction associated with a first pair of contiguous pixels at a boundary of the silhouette of the patient in the visible or infrared image of the patient and a determination of a second direction associated with a second pair of contiguous pixels at the boundary of the silhouette of the patient in the visible or infrared image of the patient. A turning point is determined when an angle between the first direction and the second direction is greater than or equal to a threshold angle.

In an example, the first pair of contiguous pixels at the boundary of the silhouette of the patient in the visible or infrared image of the patient is contiguous with the second pair of contiguous pixels at the boundary of the silhouette of the patient in the visible or infrared image of the patient.

Alternatively, in an example, the first pair of contiguous pixels at the boundary of the silhouette of the patient in the visible or infrared image of the patient share a common pixel with the second pair of contiguous pixels at the boundary of the silhouette of the patient in the visible or infrared image of the patient.

In this manner by determining feature points of a silhouette of an image of the patient, features such as arms, legs, waist, head, and chest can be differentiated from one another, and where an X-ray examination of any of these body parts is required to be taken a thickness of this body part can be determined from the silhouette of the 2D image, that could for example at a waist position show arms, but the waist itself can be identified in the silhouette from the feature points from which the thickness of the waste itself can be determined enabling the correct X-ray dose for an X-ray examination of the waste to be determined.

In an example, the determination of the thickness of the patient comprises a determination of a width of the patient perpendicular to a viewing direction of a camera that acquired the visible or infrared image of the patient comprising utilization of the silhouette of the patient in the visible or infrared image of the patient, and the processing unit is configured to transform the width of the patient to the thickness of the patient.

In an example, the transformation of the width of the patient to the thickness of the patient comprises utilization of a look up table or regression model or mathematical model.

In other words, knowledge of the width to depth relationship for people can be utilized to transform a width for example of a chest or waist in a simple 2D image to a thickness of the chest or waist as would be seen by X-rays in an X-ray examination, and this thickness can be used to determine the correct X-ray dose for the examination.

In an example, the determination of the width of the patient perpendicular to the viewing direction of the camera that acquired the visible or infrared image of the patient comprises utilization of the contour of the silhouette of the patient in the visible or infrared image of the patient.

In a second aspect, there is provided an X-ray dose determination system, comprising:
a visible or infrared camera;
a processing unit; and
an output unit.

The visible or infrared camera is configured to acquire a visible or infrared image of a patient. The visible or infrared camera is configured to provide the visible or infrared image of the patient to the processing unit. The processing unit is configured to determine a silhouette of the patient in the visible or infrared image of the patient. The processing unit is configured to determine a thickness of the patient comprising utilization of the silhouette of the patient in the visible or infrared image of the patient. The processing unit is configured to determine an X-ray dose for an X-ray examination of the patient comprising utilization of the thickness of the patient; and/or the processing unit is configured to determine that an atypical X-ray dose for the X-ray examination of the patient is required comprising utilization of the thickness of the patient. The output unit is configured to output an indication of the X-ray dose for the X-ray examination of the patient and/or the output unit is configured to output an indication that an atypical X-ray dose for the X-ray examination of the patient is required.

In a third aspect, there is provided an X-ray system, comprising:
an X-ray image acquisition unit;
a visible or infrared camera; and
a processing unit;

The visible or infrared camera is configured to acquire a visible or infrared image of a patient prior to having an X-ray examination with the X-ray image acquisition unit. The visible or infrared camera is configured to provide the visible or infrared image of the patient to the processing unit. The processing unit is configured to determine a silhouette of the patient in the visible or infrared image of the patient. The processing unit is configured to determine a thickness of the patient comprising utilization of the silhouette of the patient in the visible or infrared image of the patient. The processing unit is configured to determine an X-ray dose for the X-ray examination of the patient comprising utilization of the thickness of the patient; and/or the processing unit is configured to determine that an atypical X-ray dose for the X-ray examination of the patient is required comprising utilization of the thickness of the patient and utilize an output unit to output an indication that an atypical X-ray dose for the X-ray examination of the patient is required.

In a fourth aspect, there is provided an X-ray dose determination method, comprising:
providing a visible or infrared image of a patient to a processing unit;
determining by the processing unit a silhouette of the patient in the visible or infrared image of the patient;
determining by the processing unit a thickness of the patient comprising utilizing the silhouette of the patient in the visible or infrared image of the patient;
determining by the processing unit an X-ray dose for an X-ray examination of the patient comprising utilizing the thickness of the patient; and/or determining by the processing unit that an atypical X-ray dose for the X-ray examination of the patient is required comprising utilizing the thickness of the patient; and
outputting by an output unit an indication of the X-ray dose for the X-ray examination of the patient and/or outputting by the output unit an indication that an atypical X-ray dose for the X-ray examination of the patient is required.

In an aspect, there is provided a computer program element for controlling an apparatus according to the first aspect which when executed by a processor is configured to carry out the method of the fourth aspect.

In an aspect, there is provided a computer program element for controlling a system according to the second aspect which when executed by a processor is configured to carry out the method of the fourth aspect.

In an aspect, there is provided a computer program element for controlling a system according to the third aspect which when executed by a processor is configured to carry out the method of the fourth aspect.

Thus, according to aspects, there is provided computer program elements controlling one or more of the apparatuses/systems as previously described which, if the computer program element is executed by a processor, is adapted to perform the method as previously described.

According to another aspect, there is provided computer readable media having stored the computer elements as previously described.

The computer program element can for example be a software program but can also be a FPGA, a PLD or any other appropriate digital means.

Advantageously, the benefits provided by any of the above aspects equally apply to all of the other aspects and vice versa.

The above aspects and examples will become apparent from and be elucidated with reference to the embodiments described hereinafter.

### BRIEF DESCRIPTION OF THE DRAWINGS

Exemplary embodiments will be described in the following with reference to the following drawing:

FIG. 1 shows an example of an X-ray dose determination apparatus;

FIG. 2 shows an example of an X-ray dose determination system;

FIG. 3 shows an example of an X-ray system;

FIG. 4 shows an X-ray dose determination method; and

FIG. 5 shows an example of an image having been annotated to provide a segmentation mask to be used in training a segmentation machine learning algorithm;

FIG. 6 shows an example of direction encoding of different contour points, with specific examples of encoding equal to 1 and encoding equal to 6;

FIG. 7 shows an example of feature point determination; and

FIG. 8 shows an example of radiation dose increase with patient thickness for an X-ray imaging of the chest to yield images of equivalent image quality.

### DETAILED DESCRIPTION OF EMBODIMENTS

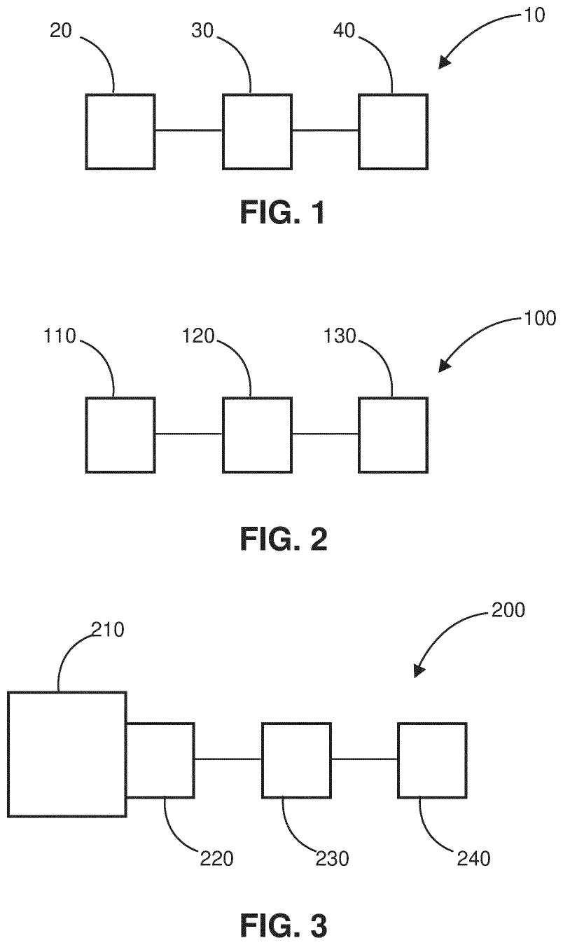

FIG. 1 shows an example of an X-ray dose determination apparatus 10. The apparatus 10 comprises an input unit 20, a processing unit 30, and an output unit 40. The input unit is configured to provide a visible or infrared image of a patient to the processing unit. The processing unit is configured to determine a silhouette of the patient in the visible or infrared image of the patient. The processing unit is configured to determine a thickness of the patient comprising utilization of the silhouette of the patient in the visible or infrared image of the patient. The processing unit is configured to determine an X-ray dose for an X-ray examination of the patient comprising utilization of the thickness of the patient. Additionally or alternatively the processing unit is configured to determine that an atypical X-ray dose for the X-ray examination of the patient is required comprising utilization of the thickness of the patient. The output unit is configured to output an indication of the X-ray dose for the X-ray examination of the patient. Additionally or alternatively the output unit is configured to output an indication that an atypical X-ray dose for the X-ray examination of the patient is required.

In this manner a simple 2D image of a person acquired by a camera can be used to determine a thickness of a patient from which a correct X-ray dose can be determined and/or an indication given to an operator that an atypical or non-typical X-ray dose will be required. In this manner, for patients who are larger than normal and/or have more body fat can be accommodated where the correct X-ray dose can be given to enable a resultant X-ray with the required contrast to be acquired and mitigate overexposure or underexposure of the patient.

In other words, a 2D image of a patient is used to determine the patient's morphology from which an X-ray dose for an X-ray examination can be determined.

To put this another way a simple camera, such as an RGB camera, can be used to acquire an image and the patient's silhouette is evaluated from this image and this used to determine a thickness of the patient from which a correct X-ray dose can be determined.

In an example, the visible or infrared image is a 2D visible or infrared image acquired by a normal 2D camera.

However, the visible or infrared image can be a 3D image acquired by a 3D camera, using time-of-flight camera, or grid distortion technology. The depth resolution for such cameras is generally less than the lateral resolution, and therefore determination of a silhouette of such a 3D image to determine a thickness of the patient and an x-ray dose determination can be more accurate than determination of a thickness of a patient from the 3D image data itself, and then determining an X-ray dose.

In an example, the indication of the X-ray dose and/or the indication that an atypical X-ray dose is required comprises an X-ray dose in numerical form presented on a VDU.

In an example, the indication of the X-ray dose and/or the indication that an atypical X-ray dose is required comprises a visual indication that a different dose to that anticipated is proposed.

In an example, the indication of the X-ray dose and/or the indication that an atypical X-ray dose is required comprises a visual representation in colour—for example with a first colour indicating that the X-ray dose is as anticipated, a second colour indicating that a higher dose than anticipated is required, and a third colour indicating that a lower dose than anticipated is required.

In an example, the indication of the X-ray dose and/or the indication that an atypical X-ray dose is required comprises a visual representation of a marker shown with respect to a scale—thus for example a scale with upper and lower boundaries annotated on it can be presented to a technician with a slider or marker that moves to the required dose following processing of the visible or infrared image.

According to an example, the determination of the silhouette of the patient in the visible or infrared image of the patient comprises implementation by the processing unit of a segmentation machine learning algorithm to analyse the visible or infrared image of the patient to determine a segmentation mask representative of the silhouette of the patient in the visible or infrared image of the patient.

According to an example, the segmentation machine learning algorithm was trained on a plurality of visible or infrared images of one or more persons and an associated plurality of segmentation masks obtained from annotation of an outline of the one or more persons, and wherein each visible or infrared image has an image of one person.

In an example, the machine learning algorithm utilizes DeepLab.

According to an example, the processing unit is configured to determine a contour of the silhouette of the patient in the visible or infrared image of the patient comprising utilization of the silhouette of the patient in the visible or infrared image of the patient. The determination of the thickness of the patient can then comprise utilization of the contour of the silhouette of the patient in the visible or infrared image of the patient.

According to an example, the processing unit is configured to determine a plurality of feature points of the silhouette of the patient in the visible or infrared image of the patient that represent the contour of the silhouette of the patient in the visible or infrared image of the patient. The determination of the thickness of the patient can then comprise utilization of the plurality of feature points of the silhouette of the patient in the visible or infrared image of the patient.

According to an example, the determination of the plurality of feature points comprises a determination of a plurality of turning points at one or more boundaries of the silhouette of the patient in the visible or infrared image of the patient, and wherein each turning point defines a feature point.

According to an example, the determination of a turning point comprises a determination of a first direction associated with a first pair of contiguous pixels at a boundary of the silhouette of the patient in the visible or infrared image of the patient and a determination of a second direction associated with a second pair of contiguous pixels at the boundary of the silhouette of the patient in the visible or infrared image of the patient, and a turning point is determined when an angle between the first direction and the second direction is greater than or equal to a threshold angle.

In an example, the threshold angle is 35 degrees, or 40 degrees, or 45 degrees, or 50 degrees, or 55 degrees, or 60 degrees, or 65 degrees, or 70 degrees, or 75 degrees, or 80 degrees, or 85 degrees, or 90 degrees, or 95 degrees, or 100 degrees, or 105 degrees, or 110 degrees.

According to an example, the first pair of contiguous pixels at the boundary of the silhouette of the patient in the visible or infrared image of the patient is contiguous with the second pair of contiguous pixels at the boundary of the silhouette of the patient in the visible or infrared image of the patient. Alternatively, according to an example the first pair of contiguous pixels at the boundary of the silhouette of the patient in the visible or infrared image of the patient share a common pixel with the second pair of contiguous pixels at the boundary of the silhouette of the patient in the visible or infrared image of the patient.

In this manner by determining feature points of a silhouette of an image of the patient, features such as arms, legs, waist, head, and chest can be differentiated from one another, and where an X-ray examination of any of these body parts is required to be taken a thickness of this body part can be determined from the silhouette of the 2D image, that could for example at a waist position show arms, but the waist itself can be identified in the silhouette from the feature points from which the thickness of the waste itself can be determined enabling the correct X-ray dose for an X-ray examination of the waste to be determined.

According to an example, the determination of the thickness of the patient comprises a determination of a width of the patient perpendicular to a viewing direction of a camera that acquired the visible or infrared image of the patient comprising utilization of the silhouette of the patient in the visible or infrared image of the patient, and the processing unit is configured to transform the width of the patient to the thickness of the patient.

According to an example, the transformation of the width of the patient to the thickness of the patient comprises utilization of a look up table or regression model or mathematical model.

In other words, knowledge of the width to depth relationship for people can be utilized to transform a width for example of a chest or waist in a simple 2D image to a thickness of the chest or waist as would be seen by X-rays in an X-ray examination, and this thickness can be used to determine the correct X-ray dose for the examination.

In an example, the look up table is associated with a body part to be subject of the X-ray examination of the patient.

In an example, the look up table takes into account component parts and/or organs of and/or within the body part.

In an example, the regression model is associated with a body part to be subject of the X-ray examination of the patient.

In an example, the regression takes into account component parts and/or organs of and/or within the body part.

In an example, the mathematical is associated with a body part to be subject of the X-ray examination of the patient.

In an example, the mathematical model takes into account component parts and/or organs of and/or within the body part.

Thus, the thickness of the body part, such as a leg, chest, stomach can be determined from a visible or infrared image, and from knowledge of the body part and the expected amount of other structures, such as bone, or organs, and how much these structures absorb X-rays, a more accurate determination of the required X-ray dose can be determined that can also take into account how much body fat is present in addition to organs/bone.

According to an example, the determination of the width of the patient perpendicular to the viewing direction of the camera that acquired the visible or infrared image of the patient comprises utilization of the contour of the silhouette of the patient in the visible or infrared image of the patient.

In an example, the determination of the X-ray dose for the X-ray examination of the patient comprises utilization of a correlation between X-ray doses and patient thicknesses.

FIG. 2 shows an example of an X-ray dose determination system 100. The system 100 comprises a visible or infrared camera 110, a processing unit 120, and an output unit 130. The visible or infrared camera is configured to acquire a visible or infrared image of a patient. The visible or infrared camera is configured to provide the visible or infrared image of the patient to the processing unit. The processing unit is configured to determine a silhouette of the patient in the visible or infrared image of the patient. The processing unit is configured to determine a thickness of the patient comprising utilization of the silhouette of the patient in the visible or infrared image of the patient. The processing unit is configured to determine an X-ray dose for an X-ray examination of the patient comprising utilization of the thickness of the patient. Additionally or alternatively the processing unit is configured to determine that an atypical X-ray dose for the X-ray examination of the patient is required comprising utilization of the thickness of the patient. The output unit is configured to output an indication of the X-ray dose for the X-ray examination of the patient. Additionally or alternatively the output unit is configured to output an indication that an atypical X-ray dose for the X-ray examination of the patient is required.

In an example, the visible or infrared image is a 2D image, acquired by a 2D camera.

However, the visible or infrared image can be a 3D image, acquired by a 3D camera that uses technology such as time-of-flight or grid distortion.

In an example, the determination of the silhouette of the patient in the visible or infrared image of the patient comprises implementation by the processing unit of a segmentation machine learning algorithm to analyse the visible or infrared image of the patient to determine a segmentation mask representative of the silhouette of the patient in the visible or infrared image of the patient.

In an example, the segmentation machine learning algorithm was trained on a plurality of visible or infrared images of one or more persons and an associated plurality of segmentation masks obtained from annotation of an outline of the one or more persons, and each visible or infrared image has an image of one person.

In an example, the machine learning algorithm utilizes DeepLab.

In an example, the processing unit is configured to determine a contour of the silhouette of the patient in the visible or infrared image of the patient comprising utilization of the silhouette of the patient in the visible or infrared image of the patient. The determination of the thickness of the patient can comprise utilization of the contour of the silhouette of the patient in the visible or infrared image of the patient.

In an example, the processing unit is configured to determine a plurality of feature points of the silhouette of the patient in the visible or infrared image of the patient that represent the contour of the silhouette of the patient in the visible or infrared image of the patient. The determination of the thickness of the patient can comprise utilization of the plurality of feature points of the silhouette of the patient in the visible or infrared image of the patient.

In an example, the determination of the plurality of feature points comprises a determination of a plurality of turning points at one or more boundaries of the silhouette of the patient in the visible or infrared image of the patient, and wherein each turning point defines a feature point.

In an example, the determination of a turning point comprises a determination of a first direction associated with a first pair of contiguous pixels at a boundary of the silhouette of the patient in the visible or infrared image of the patient and a determination of a second direction associated with a second pair of contiguous pixels at the boundary of the silhouette of the patient in the visible or infrared image of the patient, and a turning point is determined when an angle between the first direction and the second direction is greater than or equal to a threshold angle.

In an example, the threshold angle is 35 degrees, or 40 degrees, or 45 degrees, or 50 degrees, or 55 degrees, or 60 degrees, or 65 degrees, or 70 degrees, or 75 degrees, or 80 degrees, or 85 degrees, or 90 degrees, or 95 degrees, or 100 degrees, or 105 degrees, or 110 degrees.

In an example, the first pair of contiguous pixels at the boundary of the silhouette of the patient in the visible or infrared image of the patient is contiguous with the second pair of contiguous pixels at the boundary of the silhouette of the patient in the visible or infrared image of the patient.

In an example, the first pair of contiguous pixels at the boundary of the silhouette of the patient in the visible or infrared image of the patient share a common pixel with the second pair of contiguous pixels at the boundary of the silhouette of the patient in the visible or infrared image of the patient.

In an example, the determination of the thickness of the patient comprises a determination of a width of the patient perpendicular to a viewing direction of a camera that acquired the visible or infrared image of the patient comprising utilization of the silhouette of the patient in the visible or infrared image of the patient, and the processing unit is configured to transform the width of the patient to the thickness of the patient.

In an example, the transformation of the width of the patient to the thickness of the patient comprises utilization of a look up table or regression model or mathematical model.

In an example, the look up table is associated with a body part to be subject of the X-ray examination of the patient.

In an example, the look up table takes into account component parts and/or organs of and/or within the body part.

In an example, the regression model is associated with a body part to be subject of the X-ray examination of the patient.

In an example, the regression takes into account component parts and/or organs of and/or within the body part.

In an example, the mathematical is associated with a body part to be subject of the X-ray examination of the patient.

In an example, the mathematical model takes into account component parts and/or organs of and/or within the body part.

In an example, the determination of the width of the patient perpendicular to the viewing direction of the camera that acquired the visible or infrared image of the patient comprises utilization of the contour of the silhouette of the patient in the visible or infrared image of the patient.

In an example, the determination of the X-ray dose for the X-ray examination of the patient comprises utilization of a correlation between X-ray doses and patient thicknesses.

FIG. 3 shows an example of an X-ray system 200. The system comprises an X-ray image acquisition unit 210, a visible or infrared camera 220, and a processing unit 230. The visible or infrared camera is configured to acquire a visible or infrared image of a patient prior to having an X-ray examination with the X-ray image acquisition unit. The visible or infrared camera is configured to provide the visible or infrared image of the patient to the processing unit. The processing unit is configured to determine a silhouette of the patient in the visible or infrared image of the patient. The processing unit is configured to determine a thickness of the patient comprising utilization of the silhouette of the patient in the visible or infrared image of the patient. The processing unit is configured to determine an X-ray dose for the X-ray examination of the patient comprising utilization of the thickness of the patient. Additionally or alternatively the processing unit is configured to determine that an atypical X-ray dose for the X-ray examination of the patient is required comprising utilization of the thickness of the patient and utilize an output unit to output an indication that an atypical X-ray dose for the X-ray examination of the patient is required.

In an example, the visible or infrared image is a 2D image, acquired by a 2D camera.

However, the visible or infrared image can be a 3D image, acquired by a 3D camera that uses technology such as time-of-flight or grid distortion.

In an example, the determination of the silhouette of the patient in the visible or infrared image of the patient comprises implementation by the processing unit of a segmentation machine learning algorithm to analyse the visible or infrared image of the patient to determine a segmentation mask representative of the silhouette of the patient in the visible or infrared image of the patient.

In an example, the segmentation machine learning algorithm was trained on a plurality of visible or infrared images of one or more persons and an associated plurality of segmentation masks obtained from annotation of an outline of the one or more persons, and wherein each visible or infrared image has an image of one person.

In an example, the machine learning algorithm utilizes DeepLab.

In an example, the processing unit is configured to determine a contour of the silhouette of the patient in the visible or infrared image of the patient comprising utilization of the silhouette of the patient in the visible or infrared image of the patient. The determination of the thickness of the patient can comprise utilization of the contour of the silhouette of the patient in the visible or infrared image of the patient.

In an example, the processing unit is configured to determine a plurality of feature points of the silhouette of the patient in the visible or infrared image of the patient that represent the contour of the silhouette of the patient in the visible or infrared image of the patient. The determination of the thickness of the patient can comprise utilization of the plurality of feature points of the silhouette of the patient in the visible or infrared image of the patient.

In an example, the determination of the plurality of feature points comprises a determination of a plurality of turning points at one or more boundaries of the silhouette of the patient in the visible or infrared image of the patient, and wherein each turning point defines a feature point.

In an example, the determination of a turning point comprises a determination of a first direction associated with a first pair of contiguous pixels at a boundary of the silhouette of the patient in the visible or infrared image of the patient and a determination of a second direction associated with a second pair of contiguous pixels at the boundary of the silhouette of the patient in the visible or infrared image of the patient, and a turning point is determined when an angle between the first direction and the second direction is greater than or equal to a threshold angle.

In an example, the threshold angle is 35 degrees, or 40 degrees, or 45 degrees, or 50 degrees, or 55 degrees, or 60 degrees, or 65 degrees, or 70 degrees, or 75 degrees, or 80 degrees, or 85 degrees, or 90 degrees, or 95 degrees, or 100 degrees, or 105 degrees, or 110 degrees.

In an example, the first pair of contiguous pixels at the boundary of the silhouette of the patient in the visible or infrared image of the patient is contiguous with the second pair of contiguous pixels at the boundary of the silhouette of the patient in the visible or infrared image of the patient.

In an example, the first pair of contiguous pixels at the boundary of the silhouette of the patient in the visible or infrared image of the patient share a common pixel with the second pair of contiguous pixels at the boundary of the silhouette of the patient in the visible or infrared image of the patient.

In an example, the determination of the thickness of the patient comprises a determination of a width of the patient perpendicular to a viewing direction of a camera that acquired the visible or infrared image of the patient comprising utilization of the silhouette of the patient in the visible or infrared image of the patient, and the processing unit is configured to transform the width of the patient to the thickness of the patient.

In an example, the transformation of the width of the patient to the thickness of the patient comprises utilization of a look up table or regression model or mathematical model.

In an example, the look up table is associated with a body part to be subject of the X-ray examination of the patient.

In an example, the look up table takes into account component parts and/or organs of and/or within the body part.

In an example, the regression model is associated with a body part to be subject of the X-ray examination of the patient.

In an example, the regression takes into account component parts and/or organs of and/or within the body part.

In an example, the mathematical is associated with a body part to be subject of the X-ray examination of the patient.

In an example, the mathematical model takes into account component parts and/or organs of and/or within the body part.

In an example, the determination of the width of the patient perpendicular to the viewing direction of the camera that acquired the visible or infrared image of the patient comprises utilization of the contour of the silhouette of the patient in the visible or infrared image of the patient.

In an example, the determination of the X-ray dose for the X-ray examination of the patient comprises utilization of a correlation between X-ray doses and patient thicknesses.

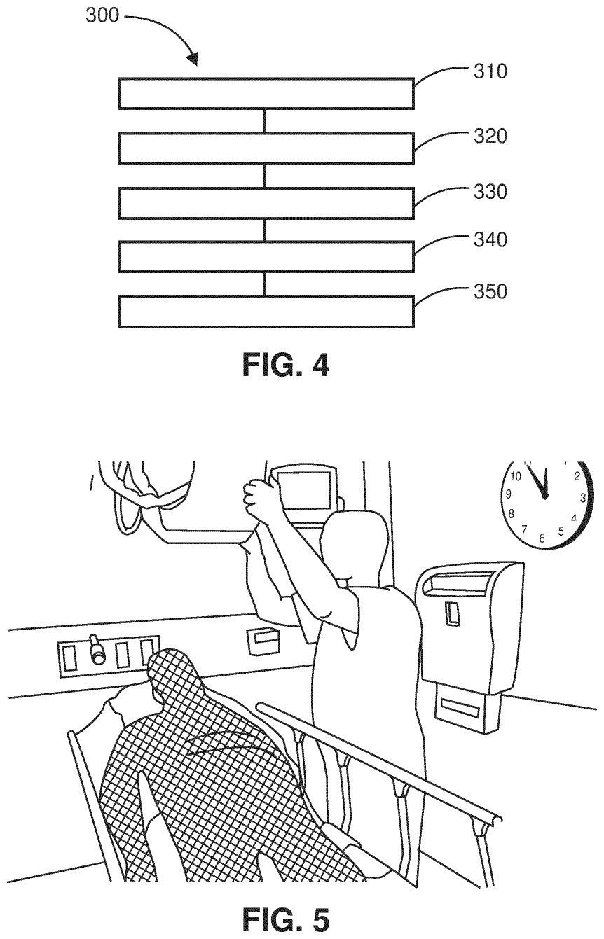

FIG. 4 shows an X-ray dose determination method 300 in its basic steps. The method 300 comprises:
providing 310 a visible or infrared image of a patient to a processing unit;
determining 320 by the processing unit a silhouette of the patient in the visible or infrared image of the patient;
determining 330 by the processing unit a thickness of the patient comprising utilizing the silhouette of the patient in the visible or infrared image of the patient;
determining 340 by the processing unit an X-ray dose for an X-ray examination of the patient comprising utilizing the thickness of the patient; and/or determining by the processing unit that an atypical X-ray dose for the X-ray examination of the patient is required comprising utilizing the thickness of the patient; and
outputting 350 by an output unit an indication of the X-ray dose for the X-ray examination of the patient and/or outputting by the output unit an indication that an atypical X-ray dose for the X-ray examination of the patient is required.

In an example, the visible or infrared image is a 2D image, acquired by a 2D camera.

However, the visible or infrared image can be a 3D image, acquired by a 3D camera that uses technology such as time-of-flight or grid distortion.

In an example, the determining the silhouette of the patient in the visible or infrared image of the patient comprises implementing by the processing unit a segmentation machine learning algorithm to analyse the visible or infrared image of the patient to determine a segmentation mask representative of the silhouette of the patient in the visible or infrared image of the patient.

In an example, the segmentation machine learning algorithm was trained on a plurality of visible or infrared images of one or more persons and an associated plurality of segmentation masks obtained from annotation of an outline of the one or more persons, and wherein each visible or infrared image has an image of one person.

In an example, the machine learning algorithm utilizes DeepLab.

In an example, the method comprises determining by the processing unit a contour of the silhouette of the patient in the visible or infrared image of the patient comprising utilizing the silhouette of the patient in the visible or infrared image of the patient. The determining the thickness of the patient can comprise utilizing the contour of the silhouette of the patient in the visible or infrared image of the patient.

In an example, the method comprises determining by the processing unit a plurality of feature points of the silhouette of the patient in the visible or infrared image of the patient that represent the contour of the silhouette of the patient in the visible or infrared image of the patient. The determining the thickness of the patient can comprise utilizing the plurality of feature points of the silhouette of the patient in the visible or infrared image of the patient.

In an example, the determining the plurality of feature points comprises determining a plurality of turning points at one or more boundaries of the silhouette of the patient in the visible or infrared image of the patient, and wherein each turning point defines a feature point.

In an example, the determining a turning point comprises determining a first direction associated with a first pair of contiguous pixels at a boundary of the silhouette of the patient in the visible or infrared image of the patient and determining a second direction associated with a second pair of contiguous pixels at the boundary of the silhouette of the patient in the visible or infrared image of the patient, and a turning point is determined when an angle between the first direction and the second direction is greater than or equal to a threshold angle.

In an example, the threshold angle is 35 degrees, or 40 degrees, or 45 degrees, or 50 degrees, or 55 degrees, or 60 degrees, or 65 degrees, or 70 degrees, or 75 degrees, or 80 degrees, or 85 degrees, or 90 degrees, or 95 degrees, or 100 degrees, or 105 degrees, or 110 degrees.

In an example, the first pair of contiguous pixels at the boundary of the silhouette of the patient in the visible or infrared image of the patient is contiguous with the second pair of contiguous pixels at the boundary of the silhouette of the patient in the visible or infrared image of the patient.

In an example, the first pair of contiguous pixels at the boundary of the silhouette of the patient in the visible or infrared image of the patient share a common pixel with the second pair of contiguous pixels at the boundary of the silhouette of the patient in the visible or infrared image of the patient.

In an example, the determining the thickness of the patient comprises determining a width of the patient perpendicular to a viewing direction of a camera that acquired the visible or infrared image of the patient comprising utilizing the silhouette of the patient in the visible or infrared image of the patient, and the method comprises transforming by the processing unit the width of the patient to the thickness of the patient.

In an example, the transforming the width of the patient to the thickness of the patient comprises utilizing a look up table or a regression model or mathematical model.

In an example, the look up table is associated with a body part to be subject of the X-ray examination of the patient.

In an example, the look up table takes into account component parts and/or organs of and/or within the body part.

In an example, the regression model is associated with a body part to be subject of the X-ray examination of the patient.

In an example, the regression takes into account component parts and/or organs of and/or within the body part.

In an example, the mathematical is associated with a body part to be subject of the X-ray examination of the patient.

In an example, the mathematical model takes into account component parts and/or organs of and/or within the body part.

In an example, the determining the width of the patient perpendicular to the viewing direction of the camera that acquired the visible or infrared image of the patient comprises utilizing the contour of the silhouette of the patient in the visible or infrared image of the patient.

In an example, the determination of the X-ray dose for the X-ray examination of the patient comprises utilization of a correlation between X-ray doses and patient thicknesses.

The X-ray dose determination apparatus, the X-ray dose determination system, the X-ray system, and the X-ray dose determination method are now described in specific details, where reference is made to FIGS. 5-8.

The following relates to utilization of a 2D visible or infrared image. However, the visible or infrared image can be a 3D image acquired by a 3D camera, using time-of-flight camera, or grid distortion technology, and the discussion below with respect to a 2D image can also apply to a 3D image.

The X-ray dose determination system, the X-ray system, and the X-ray dose determination method provides that the patient's position and body type is evaluated via a 2D simple image in order to determine a thickness to enable an X-ray radiation dose to be determined such that kV and mAs parameters can be determined, that will result in a better-quality X-ray image. By using a 2D image to estimate a patient's body type and thickness with respect to the subsequent passage of X-rays, a radiation dose can be determined that helps technicians to choose the right voltage (kV) and radiation intensity (mAs) parameters for optimal X-ray procedures, and for a fixed kV the radiation intensity (mAs) can be automatically determined. By avoiding under-exposed images, the number of retakes can be reduced, which ultimately lowers device exploitation costs and patient X-ray exposure. By avoiding over-exposed images, patient exposure is reduced, which ultimately translates in better patient outcomes.

It was realized by the inventors that human bodies have certain scale similarities, where for example there is a relationship between a width at the waist and a depth of the person (perpendicular to the width) at the waist and a relationship between a width at the chest and a depth of the person (perpendicular to the width) at the waist, and such scaling can apply across the body parts. This led to the realization that a simple 2D image of a patient could be utilized to determine a thickness of the patient, as would be seen by X-rays passing through the body, for a part of the body to be the subject of an X-ray examination to acquire an x-ray image. This thickness could then be used to determine the X-ray dose that would lead to an image quality commensurate with examination, without overexposure or underexposure occurring.

Thus, one or more photographs of a patient, obtained for example by a RGB camera associated with an X-ray image acquisition unit, are evaluated by a processing unit, that in effect acts like a Body Type Evaluation Module, that based on the one or more photographs determines a thickness through the patient as would be seen by X-rays. This enables a radiation dose for the patient to be determined. From details such as the height of the patient and other features such as the width of the patient's chest, waist, and other body parts not only can the thickness through the patient be determined but the thickness of a layers of fat that X-ray would propagate through can be determined, that enables a more accurate determination of radiation dose to be determined: in effect the fundamental body type of the patient can be determined and this enables the thickness of the body and what fat thickness makes up that thickness to be determined. However, a determination of just the thickness through the patient without determining a fat layer can itself be used to determine an X-ray dose for the patient that will lead to a satisfactory X-ray image. Thus, the determined dose or determined thickness of the patient, at the location where the X-ray examination is to be made, can be notified to a technician who could then utilize the dose level provided to them or realise that a different dose level than would be normally used is now required, such as a higher dose for a patient who is fatter than normal. The determined dose itself can be automatically provided to the X-ray imaging unit to automatically adjust the dose level as required, without interaction from the technician. As is becoming more common, cameras with time of flight functionality or grid distortion that can acquire 3d imagery can also be utilized rather than a simple 2D visible or infrared camera.

The processing unit, acting as the Body Type Evaluation Module, can be calibrated to convert pixel sizes as seen by the camera to physical measurements, like thickness of tissue to be traversed by X-Ray. This calibration can be achieved by utilization of an object of known physical dimensions to be placed near the patient during the study (an ‘etalon’). This enables the camera to be moved with respect to the patient, in terns of being closer or further away. However, if the angular extent of pixels is known, then a simple determination of distance to the patient provides a calibration, and if the camera is utilized at a fixed distance, then calibration is only required to be done once. There are many such ways to calibrate a 2D camera image such that actual sizes of imaged objects can be determined. The above discussion also applies to an infrared camera and not just to an RGB camera, or indeed to a greyscale camera.

Thus, preliminary to an X-ray) a photo of patient is taken with a RGB camera and with the help of AI a recommendation for the following X-ray study in terms of a required dose is predicted. A segmentation network, such as using DepLabV3, is used to obtain the patient's silhouette in the photo (or image) acquired by the RGB camera. The segmentation network can be trained using an annotated dataset. Once the patient's silhouette has been obtained, specific key points can be determined on it, which allows to evaluate desired measurements, such as if the patient is lying flat, the distance between the tip of his head and the end of his legs, and most importantly the thickness through the patient and the patient's fat layer. Once the thickness through the patient, and optionally patient's fat layer, has been evaluated, a warning for “high patient body habitus” can be displayed on screen for the technician to adapt the dose if the thickness is greater than would be expected. Alternatively or additionally, the required dose can be adapted automatically using precomputed look-up tables or other means.

A hybrid AI segmentation algorithm, using a third version of DeepLab, is used to extract a silhouette of the patient from the 2D image acquired by the RGB camera. The segmentation of the patient's silhouette, while lying in ward beds made use of classical supervised machine learning tasks, similar to the segmentation of road scenes in automated driving or lung segmentation, see for instance L-C. Chen, G. Papandreou, F. Schroff, H. Adam, Rethinking Atrous Convolution for Semantic Image Segmentation, arXiv cs.CV, available online: https://arxiv.org/abs/1706.05587v3. To train the hybrid AI segmentation algorithm an annotated data set of 2D camera imagery of patients or persons is used. An example of an annotated image is shown in FIG. 5, where pixels in the image have been identified as either relating to the patient or not relating to the patient. This annotated image is in effect a segmentation mask that is also a silhouette of the patient. Both images are then used, along with multiple examples of other 2D images and their annotated counterpart, to train the hybrid AI segmentation algorithm. Thus, when a 2D image of a patient on a bed, who is being prepared for an X-ray examination, is acquired and fed into the hybrid AI segmentation algorithm a segmentation mask that provides a silhouette of the patient is output.

Thus for this training, pairs of two images are used: the source image and the segmentation mask. The mask is obtained from the annotation of the source image, that at a simplistic level could be obtained by the source image photograph in paint and drawing the contour of the patient object with the mouse. The mask is created from this annotation: it is an image of the same size as the source image. The mask is entirely black, save for the pixels of the boundary and the pixels laying inside of the boundary. The image is given at the input of the Fully Convolutional Neural Network (FCN), that can utilize DeepLab or another algorithm, and a result is obtained. This result is compared with the mask, aiming at the minimization of the L1 loss: the sum of the absolute difference of each pixel. Another popular choice would be the L2 loss: the sum of the square differences. The maximization of the Dice Score (also named Intersection over Union, IoU) is possible as well. The errors are used to modify the weights of the neural network by backpropagation and gradient descent.

Then during the inference phase, where a segmentation mask is required for a new image, only the image is given at the input of the neural network, and the mask is obtained at its output.

Thus, in the specific example described here, images of patients acquired by an RGB camera are annotated to define the silhouette of the patient on the images. Afterwards, DeepLabV3 is trained, and finally the silhouette of new patients is obtained by giving the image to the trained DeepLab algorithm.

Reference is made to utilization of DeepLab, however other networks such as UNet could be utilized.

DeepLab is a family of Fully Convolutional Neural networks, that are extremely efficient for image semantic segmentation. The first version of DeepLab appeared around the same time with UNet (2015), with which it shares the overall “encoder-decoder” architecture. There are key differences however.

UNet has a symmetric encoder-decoder architecture, meaning the size of the intermediate tensors mirror each other at the different stages. Moreover UNet has a concatenation mechanism, that helps obtaining segmentation results, that closely match the boundary of the objects on the original image.

DeepLab on the contrary has a massive encoder and a small decoder, called “projection head”. It is commonplace to train a large classification network, such as ResNet101, on the ImageNet dataset and then use it as an encoder in DeepLab. In the third version of DeepLab, Atrous Spatial Pyramid Pooling (ASPP) is utilized that deploys inverse transposed convolutions at different scales in order to match the features extracted by the encoder to the images. The results are segmentation mask, that match closely in shape the object to which they correspond, and is why DeepLab V3 was utilized, but other algorithms can be utilized such as UNet.

Thus, the hybrid AI algorithm is used to extract the patient's silhouette and evaluate the thickness through the patient and a thickness of fat layers. This technique is applicable to both facing and profile photos, and can be applied for patient laying in prone, supine, or lateral decubitus positions.

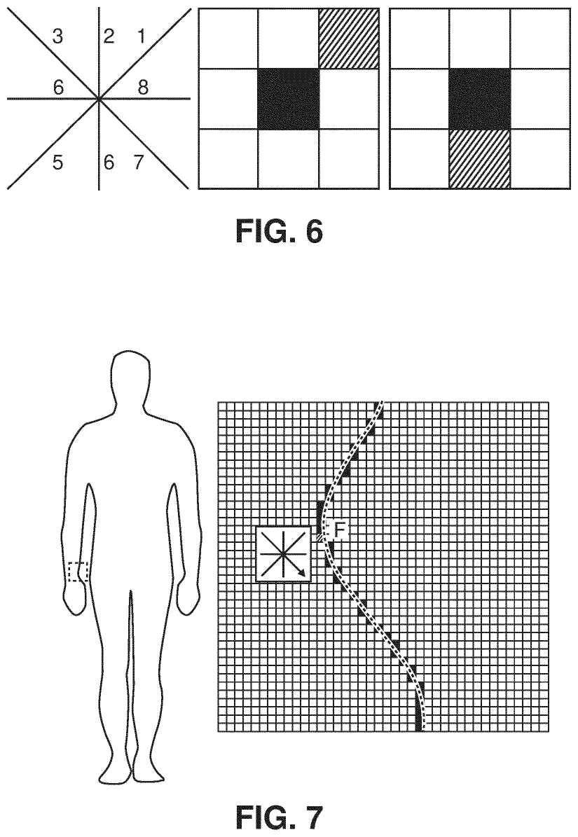

However, rather than just utilize the silhouette of the patient as such, feature points of the silhouette are extracted, that define the silhouette and enable parts of the body to be differentiated from each other—arms from the body truck etc, from which an accurate thickness at the chest and waist can be determined. This is done as follows, with reference to FIGS. 6 and 7.

Once the silhouette is extracted, the silhouette contour is obtained. A starting point on the edge of the silhouette is chosen arbitrarily, and every point •. of the contour is transformed into a sequence of numbers, based on the direction of the neighboring pixel. The number coding of the neighboring pixel is shown in the first diagram of FIG. 6, where here directions have been numbered as 0 to 8 at 45 degree intervals. In the second diagram of FIG. 6 the direction of the neighboring pixel is 1 and the direction of the following pixels is shown in the third diagram of FIG. 6 and the direction is 6. Therefore, the coding •. for this part of the contour is 3, and 6.

Once the sequence of neighboring directions •. has been obtained for the boundary of the silhouette, it can be used to create the sequence of turning points. by applying the relation:
•.••.••.•

That is a turning point is defined by the fact that the direction of its neighboring pixels differs from that of the starting point.

Finally, the features points •. can be defined using the relation
•.•|•...••.|=•

That is a Feature point •. is defined when the contour's direction of turning is 90° for a value of X equal to 2, however other values can be utilized and an average direction can be calculated for different parts of the boundary or contour of the silhouette. This process is shown in FIG. 7.

The feature points then define or encode the patient's body shape.

At this stage, the distance between feature points is known in terms of pixels. In order to translate those pixel measurements into physical quantities, an etalon can be used. An etalon any object of known physical dimensions that is imaged together with the patient. A good candidate for an etalon would be the L/R marker that the technician already has to insert on each X-ray in order to indicate the patient's left, respectively right, side. By requiring that the marker always be physical and of predefined size, this marker can be turned into an etalon.

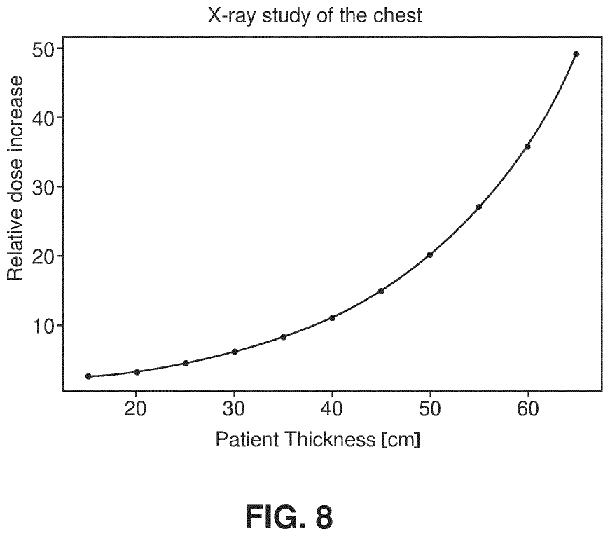

The feature points enable an accurate width of different parts of the patients body, such as waist and chest to be determined, and from this width a thickness through the patient, for example perpendicular to the width can be determined from known values for body thickness versus a perpendicular body width. Use is then made of the an X-ray dose required as a function of body thickness to result in a satisfactory X-ray image, for example as shown in FIG. 8 that shows the relative increase in X-ray dose as a function of body thickness for a chest X-ray.

Thus, in a situation where a patient with a 40 cm thick chest is to be X-rayed a ten-fold increase in x-ray dose is required in order to acquire an X-ray image of equivalent quality as if the patient only had a 20 cm thick chest. The new technique enables this correction to be made automatically and inform the technician at the same time if necessary who could validate or initiate the required dose change, or this change could be done automatically.

As detailed above, use is made linking the patient's thickness with the patient's width.

Here, a look up table can be utilized, that could look be a two or three column table: where column 1 is for example width at the waist/chest, and column 2 is thickness at the waist/chest. Then a graph such as that shown in FIG. 8 can be utilized to provide for dose adaptation. However, the required dose adaptation for the waist/chest could be at a third column of the look up table. This is then repeated for the width at the shoulders, the width at the hip, etc. It is also possible to fit a polynomial regression or any AI regression model to enact the transformation between the width and the thickness.

In another exemplary embodiment, a computer program or computer program element is provided that is characterized by being configured to execute the method steps of any of the methods according to one of the preceding embodiments, on an appropriate apparatus or system.

The computer program element might therefore be stored on a computer unit, which might also be part of an embodiment. This computing unit may be configured to perform or induce performing of the steps of the method described above. Moreover, it may be configured to operate the components of the above-described system. The computing unit can be configured to operate automatically and/or to execute the orders of a user. A computer program may be loaded into a working memory of a data processor. The data processor may thus be equipped to carry out the method according to one of the preceding embodiments.

This exemplary embodiment of the invention covers both, a computer program that right from the beginning uses the invention and computer program that by means of an update turns an existing program into a program that uses the invention.

Further on, the computer program element might be able to provide all necessary steps to fulfill the procedure of an exemplary embodiment of the method as described above.

According to a further exemplary embodiment of the present invention, a computer readable medium, such as a CD-ROM, USB stick or the like, is presented wherein the computer readable medium has a computer program element stored on it which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfill the functions of several items re-cited in the claims. The mere fact that certain measures are re-cited in mutually different dependent claims does not indicate that a combination of these measures cannot be used to advantage. Any reference signs in the claims should not be construed as limiting the scope.

1. An X-ray dose determination apparatus comprising:
a memory that stores a plurality of instructions; and
a processor coupled to the memory and configured to execute the plurality of instructions to:
receive a visible or infrared image of a patient;
determine a silhouette of the patient in the visible or infrared image of the patient;
determine a thickness of the patient comprising utilization of the silhouette of the patient in the visible or infrared image of the patient;
determine an X-ray dose for an X-ray examination of the patient comprising utilization of the thickness of the patient; and/or determine that an atypical X-ray dose for the X-ray examination of the patient is required comprising utilization of the thickness of the patient; and
output an indication of the X-ray dose for the X-ray examination of the patient; and/or output an indication that an atypical X-ray dose for the X-ray examination of the patient is required.

2. The apparatus according to claim 1, further comprising using a segmentation machine learning algorithm to analyze the visible or infrared image of the patient to determine a segmentation mask representative of the silhouette of the patient in the visible or infrared image of the patient.

3. The apparatus according to claim 2, wherein the segmentation machine learning algorithm is trained on a plurality of visible or infrared images of one or more persons and an associated plurality of segmentation masks obtained from annotation of an outline of the one or more persons, and wherein each visible or infrared image has an image of one person.

4. The apparatus according to claim 1, wherein the processor is configured to determine a contour of the silhouette of the patient in the visible or infrared image of the patient comprising utilization of the silhouette of the patient in the visible or infrared image of the patient, and wherein the determination of the thickness of the patient comprises utilization of the contour of the silhouette of the patient in the visible or infrared image of the patient.

5. The apparatus according to claim 4, wherein the processor is configured to determine a plurality of feature points of the silhouette of the patient in the visible or infrared image of the patient that represent the contour of the silhouette of the patient in the visible or infrared image of the patient, and wherein the determination of the thickness of the patient comprises utilization of the plurality of feature points of the silhouette of the patient in the visible or infrared image of the patient.

6. The apparatus according to claim 5, wherein determination of the plurality of feature points comprises a determination of a plurality of turning points at one or more boundaries of the silhouette of the patient in the visible or infrared image of the patient, and wherein each turning point defines a feature point.

7. The apparatus according to claim 6, wherein the determination of a turning point comprises a determination of a first direction associated with a first pair of contiguous pixels at a boundary of the silhouette of the patient in the visible or infrared image of the patient and a determination of a second direction associated with a second pair of contiguous pixels at the boundary of the silhouette of the patient in the visible or infrared image of the patient, and wherein a turning point is determined when an angle between the first direction and the second direction is greater than or equal to a threshold angle.

8. The apparatus according to claim 7, wherein the first pair of contiguous pixels at the boundary of the silhouette of the patient in the visible or infrared image of the patient is contiguous with the second pair of contiguous pixels at the boundary of the silhouette of the patient in the visible or infrared image of the patient; or wherein the first pair of contiguous pixels at the boundary of the silhouette of the patient in the visible or infrared image of the patient share a common pixel with the second pair of contiguous pixels at the boundary of the silhouette of the patient in the visible or infrared image of the patient.

9. The apparatus according to claim 1, wherein determination of the thickness of the patient comprises a determination of a width of the patient perpendicular to a viewing direction of a camera that acquired the visible or infrared image of the patient comprising utilization of the silhouette of the patient in the visible or infrared image of the patient, and wherein the processor is configured to transform the width of the patient to the thickness of the patient.

10. The apparatus according to claim 9, wherein transformation of the width of the patient to the thickness of the patient comprises utilization of a look up table, a regression model, or a mathematical model.

11. The apparatus according to claim 9, wherein the determination of the width of the patient perpendicular to the viewing direction of the camera that acquired the visible or infrared image of the patient comprises utilization of the contour of the silhouette of the patient in the visible or infrared image of the patient.

12. (canceled)

13. An X-ray system, comprising:
an X-ray image acquisition device;
a visible or infrared camera; and
a processor;
wherein the visible or infrared camera is configured to acquire a visible or infrared image of a patient prior to having an X-ray examination with the X-ray image acquisition device;
wherein the visible or infrared camera is configured to provide the visible or infrared image of the patient to the processor; wherein the processor is configured to determine a silhouette of the patient in the visible or infrared image of the patient;
wherein the processor is configured to determine a thickness of the patient comprising utilization of the silhouette of the patient in the visible or infrared image of the patient;
wherein the processor is configured to determine an X-ray dose for the X-ray examination of the patient comprising utilization of the thickness of the patient; and/or the processor is configured to determine that an atypical X-ray dose for the X-ray examination of the patient is required comprising utilization of the thickness of the patient and output an indication that an atypical X-ray dose for the X-ray examination of the patient is required.

14. A computer implemented X-ray dose determination method, comprising:
providing a visible or infrared image of a patient to a processor; determining by the processor a silhouette of the patient in the visible or infrared image of the patient;
determining by the processor a thickness of the patient comprising utilizing the silhouette of the patient in the visible or infrared image of the patient;
determining by the processor an X-ray dose for an X-ray examination of the patient comprising utilizing the thickness of the patient; and/or determining by the processor that an atypical X-ray dose for the X-ray examination of the patient is required comprising utilizing the thickness of the patient; and
outputting an indication of the X-ray dose for the X-ray examination of the patient; and/or outputting an indication that an atypical X-ray dose for the X-ray examination of the patient is required.

15. (canceled)
